(19) United States
(12) Patent Application Publication
(10) Pub. No.: US 2019/0030087 A1
(43) Pub. Date: Jan. 31, 2019

(54) Composition Comprising an Extract of Liriopsis Tuber for Protecting Brain Cells and Improving Memory

(71) Applicant: Sung-Jin Kim, Seoul (KR)

(72) Inventor: Sung-Jin Kim, Seoul (KR)

(21) Appl. No.: 12/185,009

(22) Filed: Aug. 1, 2008

Related U.S. Application Data
(62) Division of application No. 10/509,150, filed on Sep. 27, 2004, now abandoned.

(30) Foreign Application Priority Data
Mar. 29, 2002 (KR) 10-2002-0017233

(51) Int. Cl. A61K 35/60; A23L 2/52; A23L 33/105

(52) U.S. Cl. CPC A61K 35/60; A23L 2/52; A23L 33/105

(57) ABSTRACT

The present invention relates to a composition comprising an extract of Liriopsis tuber for protecting brain cells or improving memory. The composition of the present invention induces protection of brain cells in men under brain damage by environmental causes such as various stress, drinking and smoking and enhancement of memory, and based on this, can be used as medicines, foodstuffs and beverages which are effective for prevention and treatment of neurodegenerative diseases and for enhancing memory.

### TECHNICAL FIELD

The present invention relates to a composition comprising an extract of

Liriopsis tuber for protecting brain cells or improving memory.

### BACKGROUND ART

One of the major factors associated with damage of brain cells is glutamate as an amino acid. Glutamate acts via combining primarily to the four receptors, i.e. NMDA (N-methyl-D-aspartate) receptor, AMPA (L-α-amino-3-hydroxy-5-methyl-4-isoxazolepropionate) receptor, Kainate receptor and 1S, 3R-ACPD receptor [Craig C R, Stitzel R E, Modern Pharmacology with Clinical Applications, p293-302, 1997]. In the case of ischemia in brain, it causes reduction of oxygen supply to the brain cells, leading to increased anaerobic glycolysis, a decline in the action of ionic pump due to the decreased level of ATP which is an energy source within tissue, increase of the extracellular potassium ion level, resulting in depolarization of neurocellular membrane. In that case, excitatory neurotransmitter is secreted, resulting in brain damage by activation of NMDA, AMPA and Kainate receptors.

Excito-toxicity by excitatory neurotransmitter is known to play a critical role via causing cell stress in the induction of pathological state such as neurodegenerative disorders including Alzheimer's disease, Parkinsonism, stroke and amyotrophic lateral sclerosis [Haloween, B., Reactive oxygen species and the central nervous system. J Neurochem. 59, p 1609-1623, 1992; Coyle, J. T. and Puttfarcken, P., Oxidative stress, glutamate, and neurodegenerative disorders. Science 262, p 689-695, 1993; Olanow, C. W., A radical hypothesis for neurodegeneration. Trends Neurosci. 16, p 439-444, 1993]. Neurodegenerative disorders in central nervous system are often accompanied by decline of memory and cognitive function. In particular, dementia is a serious problem in today's aging society, and as the causes, heritage, aging, brain lesion, environmental causes such as smoking and drinking and other complicated factors can be considered. The hippocampus of patients suffering from dementia is heavily damaged and this is closely related to the reduction of acetylcholine levels in the brain. Currently, to raise the acetylcholine level in brain, acetylcholine esterase inhibitors are clinically used in the treatment of Alzheimer's dementia. Besides, lots of studies have been conducted concerning suppression of such brain damage [Gagliardi R J, Neuroprotection, excitotoxicity and NMDA antagonists, Arq. Neuro-Psiquiatr. p 58, 2000], and for example, NMDA antagonists, AMPA antagonists, GABA agonists, intracellular calcium reducing agents, nitric oxide inhibitors, free radical scavengers, sodium channel inhibitors, glutamate release inhibitors, growth factors, acidosis, hypothermia and potassium channel activators are under development.

Though dozocyilpin (MK 801), selfotel, cerestat and dextrometorfan have been developed as NMDA antagonists, these drugs, at a low dose, induced changes of cognition, discomfort, nystagmus and hypotension and at a high dose, exhibited mental side effects such as excitation, paranoia and hallucination. In addition, NBQX has been developed as an AMPA antagonist, but industrial applicability as medicine was very low due to serious renal toxicity.

Therefore, development of a brain-protecting agent without toxicity is urgent task in this field.

Recent studies revealed that AMPA receptor might play a key role in the occurrence of Alzheimer's disease in the light of that neuronal cell damage by activation of AMPA receptor occurs selectively on basal forebrain cholinergic neurons (BFCNs) associated with Alzheimer's disease. This suggests a possibility of developing a therapeutic for Alzheimer's disease based on an AMPA antagonist [Weiss, J. H. et al., Basal forebrain cholinergic neurons are selectively vulnerable to AMPA/kainate receptor-mediated neurotoxicity. Neuroscience 60, p 659-664].

Insulin receptors are mainly involved in glucose metabolism in peripheral tissue, while in central nervous system, it plays an important role in regulation of neuroactivity such as memory control rather than glucose metabolism. In fact, insulin receptors are widely distributed in various regions of brain tissue, in particular, dominant in hippocampus. Therefore, hippocampus becomes a primary target with respect to the role of insulin in central nervous system. Lately, many studies showed that insulin and activation of insulin receptor play a major role for memory formation in brain [Park, C. P., Seeley, R. J., Craft, S, and Woods, S. C. (2000), Intracerebroventricular insulin enhances memory in a passive avoidance task. Physiol. Behav., 68, 509-514; Zhao, W., Chen, H., Xu, H., Moore, E., Meiri, N., Quon, M. J., Alkon, D. L., (1999), Brain insulin receptors and spatial memory, J. Biol. Chem., 274, 34893-34902].

Further, ERK (extracellular signal-regulated kinase) I/II are essential signal transduction proteins connecting growth factor-mediated activation of plasma membrane receptor with changes in growth, differentiation and gene expression of cell, and it was reported that activation of ERK I/II in the cellular signal transduction mechanism is important for enhancing memory [Siddhanti et al., Endocrinology, 136, 4834-4841 (1995); Hipskind and Bilbe, Front Biosci., 1, D804-816 (1998); Thiels, E, Klann, E. Extracellular signal-regulated kinase, synaptic plasticity, and memory, Rev. Neurosci. 12, 327-345 (2001); Sweat J. D. The neuronal MAP kinase cascade: a biochemical signal integration system subserving synaptic plasticity and memory, J. Neurochem. 76, 1-10, (2001)].

Accordingly, it is considered that materials inducing the activation of insulin receptors and ERK I/II could be used for memory enhancement or dementia medicines in addition to cholinesterase inhibitors.

The inventor of the present invention has continued studies on the substance which can induce brain cells protecting effect and memory-improving effect in men suffering from brain damage by environmental causes such as stress, drinking and smoking, and as a result, discovered that an extract of Liriopsis tuber exhibits a superior effect on protecting brain cells and improving memory, and based on this, completed the present invention.

Therefore, the object of the present invention is to provide a composition comprising an extract of Liriopsis tuber for protecting brain cells or enhancing memory.

### DISCLOSURE OF INVENTION

The present invention relates to a composition comprising an extract of Liriopsis tuber for protecting brain cells or improving memory.

The composition of the present invention for protecting brain cells or improving memory, includes a Liriopsis tuber extract by 0.5 to 50% by weight based on the total weight of the composition.

Liriopsis tuber is a perennial herb classified into Liliaceae, and includes Liriope platyphylla Wang et Tang, Ophiopogon japonicus Ker-Gawl., O. stolonifer Levl. et Vant., Mondo japonicum (L.f.) Farwell and Liriope spicata (Thunb.) Lout, and swelling part of root is used for medicinal purposes. It contains components such as glucose, fructose, sucrose, ophiopogonone A and B, methylopiogonone A and B, ophiopogonanone A, methylopiopogonanone A and B, homoisoflavonoid I˜V, borneol glycoside, β-sitosterol, stigmasterol, β-sitosterol glycoside, oligosaccharides, polysaccharides, 3-O-α-L-rhamnopyranosyl(1-2)-β-D-glucopyranosylophiogenin, 5,7-dihydroxy-6-formyl-8-methyl-3-(3,4-methylenedioxybenzyl)chroman-4-one, 6-aldehydo-isoophiopogonanone A, 6-aldehydo-isoophiopogonone A, 6-aldehydo-isoophiopogonone B, 6-aldehydo-ophiopogonone A, 7-O-α-L-arabinothranosyl(1-6)-β-D-glucopyranosylbomeol, 7-O-β-D-glucopyranosyl-borneol, azetidine-2-carboxylic acid, daucosterol, methylophiopogonanone A, methylophiopogonanone B, mono-O-acetylophiopogonin D, ophiopogon C, ophiopogon amide VI, ophiopogon homoisoflavonoid I, ophiopogon homoisoflavonoid II, ophiopogon homoisoflavonoid III, ophiopogon homoisoflavonoid IV, ophiopogon homoisoflavonoid V, ophiopogonanone A, ophiopogonin A, ophiopogonin B′, ophiopogonin B, ophiopogonin C′, ophiopogonin D, ophiopogonin D′, tulipanin, vicenin 2, 25(S)-ruscogenin-1-O-α-L-rhamnopyranosyl(1-2)-β-D-fucopyranoside, 25(S)-ruscogenin-1-O-β-D-xylopyranosyl(1-3)-β-D-fucopyranoside, aster saponin Hb methyl ester, Lm-2, Lm-3, Ls-2, Ls-3, Ls-4, Ls-5, Ls-6, Ls-7, ruscogenin-1-sulfate-3-O-α-L-rhamnopyranoside, 1-sulfate-3-O-α-L-rhamnopyranosyl-ruscogenin, ruscogenin-3-O-α-L-rhamnopyranoside and ruscogenin-3-O-β-D-glucopyranosyl(1-3)-α-L-rhamnopyranoside, and used in oriental medicine for suppression of cough, expectoration, nutrition, sthenia, diuresis, suppression of thirst, blood glucose regulation, xerostomia and constipation [Illustrated Dictionary of Folk Medicine by Bosup Chung and Minlcyo Shin, Younglim company p 177-178, 1998 and New Oriental Medicine Index Traditional Medicine Database (TradMed), Natural Products Research Institute of Seoul National University, revised ed., 1999].

Nevertheless, there has not yet been a report on that a Liriopsis tuber extract has an effect on protecting brain cells and improving memory.

The Liriopsis tuber extract according to the present invention can be prepared by the following method.

Extraction method 1: the Liriopsis tuber extract can be obtained by extracting with a solvent selected from the group consisting of C₁₋₄ lower alcohols or a mixture of said lower alcohols with water, acetone, chloroform, methylene chloride, ether and ethyl acetate, preferably, methanol or a mixed solvent of methanol and water in ratio of 1:0.2-1.5. The reaction temperature is 5 to 80° C., preferably 30 to 55° C., and reaction time is 15 min to 48 hr, preferably 30 min to 12 hr.

The solvent soluble fraction thus obtained contains a large amount of terpenoids and phenolic substances.

Extraction method 2: The solvent soluble fraction obtained as described in said Extraction method 1 was dissolved in a mixture of C₁₋₄ lower alcohol and water and pH was adjusted with an acid to a range of 2-4 and then extraction was further conducted using an equal amount of chloroform to obtain a chloroform soluble fraction.

Extraction method 3: The chloroform insoluble fraction of the fractions obtained according to said Extraction method 2 was treated with ammonium hydroxide to adjust pH to 9-12 and extracted with an equal amount of a mixture of chloroform-methanol, and the fraction insoluble in the chloroform-methanol mixture was further extracted with methanol to obtain a methanol soluble fraction and a methanol insoluble aqueous fraction.

At this time, a mixing ratio of the chloroform-methanol mixed solvent is preferred to be 1:0.1-1. Of the chloroform insoluble fraction, the fraction dissolved upon extraction with the mixed solvent (chloroform-methanol) contains the majority of alkaloids, and the methanol-soluble fraction of the solvent (chloroform:methanol)-insoluble fraction includes quaternary alkaloids and N-oxides.

In addition, the Liriopsis tuber extract of the present invention can undergo additional fractionation process by conventional method (Harbome J. B. Phytochemical methods: A guide to modern techniques of plant analysis, 3^{rd} Ed., pp 6-7, 1998).

The composition of the present invention comprising an extract of Liriopsis tuber can further include at least one component selected from the group consisting of pharmaceutically acceptable carriers and additives according to conventional method.

The carrier that can be included in the composition comprising the extract of Liriopsis tuber of the present invention also includes substances commonly called excipients or diluents, and for example, at least one component selected from the group consisting of lactose, dextrose, sucrose, sorbitol, mannitol, xylitol, erythritol, maltitol, starch, isomerized sugar, sugar, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, paraoxybenzoate, methylparaoxybenzoate, paraoxypropylbenzoate, talc, magnesium stearate and mineral oil, can be used.

In addition, as the additives that can be included in the composition comprising the extract of Liriopsis tuber of the present invention, at least one component selected from the group consisting of natural carbohydrates, flavors, nutrients, vitamins, mineral (electrolytes), seasonings (synthetic, natural seasonings), coloring agents, fillers (cheese, chocolate, etc.), pectic acid and its salt, alginic acid and its salt, organic acids, protective colloidal thickeners, pH regulating agents, stabilizers, preservatives, antioxidants, glycerin, alcohols, carbonizing agents and sarcocarp, can be used.

The composition comprising the extract of Liriopsis tuber of the present invention can be used via formulating into oral administration such as powders, tablets, capsules, suspensions, emulsions, syrups and aerosols; topical applications; suppositories or sterile injections.

Though the amount of the Liriopsis tuber extract used differs depending on the age, sex, body weight of patient, daily amount of 0.1 to 500 mg/kg can be administered by dividing into one to several times. Further, the administered amount of the extract of Liriopsis tuber and its fractions can be controlled according to administration route, seriousness of disease, sex, weight and age, and said amount never limits the scope of the invention in any way. The extract of Liriopsis tuber itself according to the present invention is almost free of toxicity and adverse effect so that it can be safely used even in the case of prolonged use for the prevention purpose.

The extract of Liriopsis tuber of the present invention can also be used, together with sitologically acceptable additives, for various foodstuffs, beverages, gums, teas, vitamin complexes and health foods or beverages.

In the case of a foodstuff containing the extract of Liriopsis tuber of the present invention, the content of the Liriopsis tuber extract is 0.1 to 15% by weight, preferably 1 to 10% by weight based on the total weight of the foodstuff.

In addition, in the case of a beverage containing the extract of Liriopsis tuber of the present invention, the content of the extract of Liriopsis tuber is 1 to 30 g, preferably 3 to 10 g per 100 ml of the beverage.

Also, as the sitologically acceptable additives that can be included in the foodstuff or the beverage according to the present invention, at least one component selected from the group consisting of natural carbohydrates, flavors, nutrients, vitamins, mineral (electrolytes), seasonings (synthetic or natural seasonings), coloring agents, fillers (cheese, chocolate, etc.), pectic acid and its salt, alginic acid and its salt, organic acids, protective colloidal thickeners, pH regulating agents, stabilizers, preservatives, antioxidants, glycerin, alcohols, carbonizing agents and sarcocarp, can be used.

It is preferred for said additives to be contained in a range of 0.01 to 25 parts by weight based on 100 parts by weight of the foodstuff or beverage composition.

Additionally, as natural carbohydrates, monosaccharides such as glucose and fructose; disaccharides such as maltose and sucrose; polysaccharides such as dextrin and cyclodextrin; and sugar alcohol such as xylitol, sorbitol and erythritol can be used, and it can be generally used in an amount of about 1 to 20 g, preferably, about 5 to 12 g per 100 ml of the beverage composition.

As flavors, natural flavors such as taumatin and stevia extract (e.g. rebaudioside A, glycyrrhizin etc.); and synthetic flavors such as saccharin and aspartam can be used.

The beverage composition of the present invention has no other limitation on liquid components except containing said extract of Liriopsis tuber as an essential component at the indicated ratio.

### BRIEF DESCRIPTION OF DRAWINGS

FIG. 1 represents an inhibiting effect of a Liriopsis tuber extract (fraction T) against the depolarization of nerve cell by AMPA. The value indicated (FIG. 1B) mean±standard deviation (n=5), and significance to the control group is *: P<0.05.

FIG. 2 represents an inhibiting effect of a Liriopsis tuber extracts (fractions A, C, CM and M) against the depolarization of nerve cell by AMPA. The value indicated mean±standard deviation (n=5), and significance to the control group is *: P<0.05 and **: P<0.01.

FIG. 3 shows a memory-enhancing effect of a Liriopsis tuber extract (fraction T). The value indicated mean±standard deviation (n=8), and significance to the control group is *: P<0.05.

FIG. 4 shows a memory-enhancing effect of Liriopsis tuber extracts (fractions T, A, C, CM and M). The value indicated mean±standard deviation (n=7), and significance to the control group is *: P<0.05.

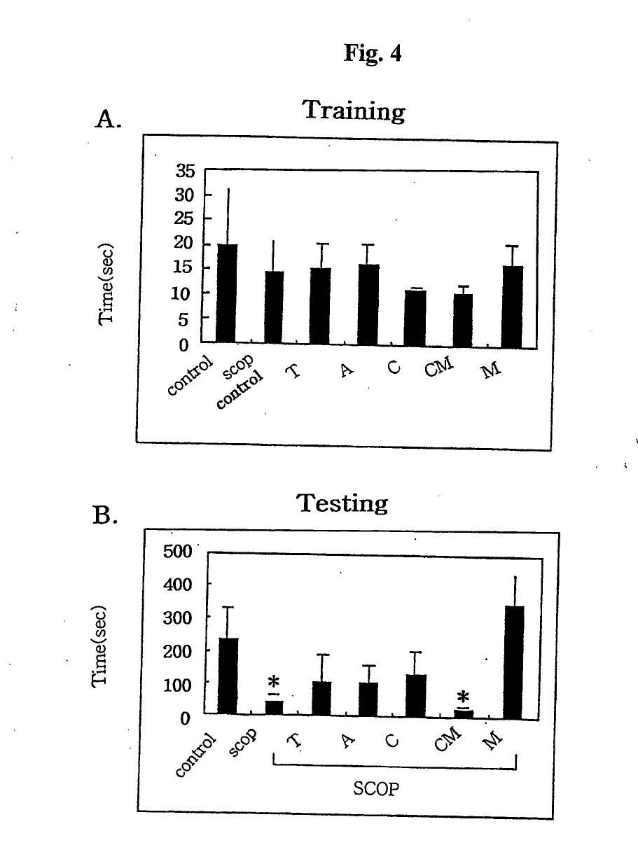

FIG. 5 demonstrates an inhibiting effect of Liriopsis tuber extracts (fractions T, A, C and M) against acetylcholine esterase. The value indicated mean±standard deviation (n=6), and significance to the control group is ***: P<0.001.

FIG. 6 represents an enhancing effect of Liriopsis tuber extracts (fractions T, A, C and M) on ERK I and ERK II activity.

FIG. 7 represents an increasing effect of Liriopsis tuber extracts (fractions T, A, C and M) on insulin receptor activity.

### BEST MODE FOR CARRYING OUT THE INVENTION

The present invention is described in more detail with the following Examples, yet they do not limit the scope of the present invention.

### EXAMPLE 1

### Preparation of an Extract of Liriopsis tuber

Liriopsis tuber 250 g was cut into small pieces and subjected to three times of extraction, each time, with 70% methanol (750 ml) using Soxhlet apparatus. The extract was filtered, subjected to concentration at a reduced pressure using a rotary evaporator (EYELA N-N series) and subjected to lyophilization to obtain a crude methanol extract (fraction T).

For further fractionation with other organic solvent, said lyophilized methanol extract 10 g was dissolved in a mixture of methanol and water (4:1) 200 ml, adjusted with 2M sulfuric acid to pH 3, extracted successively three times, each time, with an equal amount of chloroform, subjected to concentration at a reduced pressure and lyophilization to obtain a chloroform soluble fraction (fraction C), 0.12 g, and the aqueous layer was adjusted with ammonium hydroxide to pH 10, extracted two times, each time, with an equal amount of a mixture of chloroform and methanol (3:1). The chloroform-methanol (3:1) layer was subjected to concentration at a reduced pressure and lyophilization to obtain a chloroform-methanol soluble fraction (fraction CM), 0.09 g. The aqueous layer was extracted three times, each time with an equal amount of methanol, subjected to concentration at a reduced pressure and lyophilization to obtain a methanol soluble fraction (fraction M), 2.94 g and a water soluble fraction (fraction A), 2.75 g, respectively, and the fractions was used as a sample in the following activity assay.

### EXPERIMENTAL EXAMPLE 1

### Grease Gap Assay

1) Experimental Method

Wedges were prepared from cerebral cortex in white rats and placed on a two compartments brain bath, and test was performed [Harrison N L, Simmonds, M A, Quantitative studies on some antagonists of N-methyl D-aspartate in slices of rat cerebral cortex, Br. J. Pharmacol. 84, p 381-391, 1985]. Brain was quickly taken out and 2-3 mm of the front part was removed using a brain tissue slicer and then the remaining part was subjected to vertical cut to prepare a coronal section of 500-600 μm thickness and rapidly put into an oxygenated Krebs medium, and divided into two parts against median line to prepare wedges in which dorsal surface containing cerebral cortex and corpus callosum was approximately 1.5 mm wide and ventral surface was approximately 1 mm wide. After being left on the oxygenated Krebs medium for 2 hr at room temperature, the wedges were placed through a greased (high vacuum silicone grease) slot in a two compartments brain chamber. The two compartments were perfused with Krebs medium at a speed of 2 ml/min. The extracts of Liriopsis tuber (fractions T, A, C, CM and M) were perfused to the cortical end of the preparation at a concentration of 10 μg/ml for 10 min and excitatory amino acid, AMPA (α-amino-3-hydroxy-5-methyl-4-isoxazole propionic acid) (40 μM) was then applied by 2 min superfusion, and d.c. potential between the two compartments was measured with Ag/AgCl electrode, amplified by amplifier, and determined with McLab Data Acquisition System. Control experiments were also performed with AMPA only.

2) Experimental Result

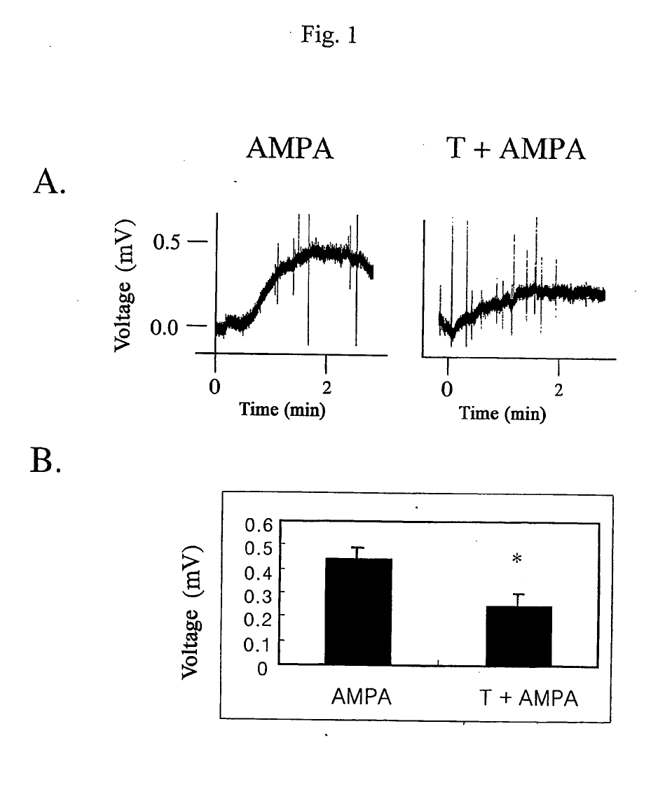

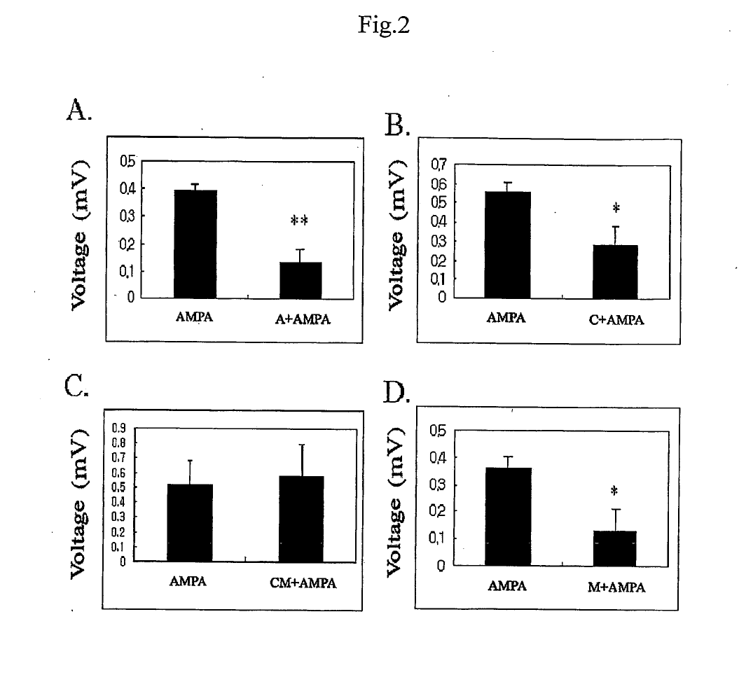

Induction of depolarization of nerve cell by AMPA is considered as a barometer of stimulus by nerve cell lesion. As a result of experiment, as can be seen from FIGS. 1A and 1B, application of AMPA (40 μM) induced depolarization of 0.44 mV, while application of AMPA after pretreatment with a Liriopsis tuber extract (fraction T) (10 μg/ml) markedly reduced the level of depolarization to 0.24 mV. In particular, pretreatments with other fractions of Liriopsis tuber extracts (fractions A, C and M) demonstrated an inhibiting effect against depolarization by AMPA, i.e. 66%, 48% and 63%, respectively (FIG. 2).

Therefore, it can be seen that the nerve protecting effect is induced by various components in the extracts of Liriopsis tuber.

### EXPERIMENTAL EXAMPLE 2

### NaNO₂ Memory Test

It is known that oxygen metabolism deficiency of brain by NaNO₂ is closely associated with cholinergic neurotransmission related with memory and learning [Schindler et al., Nootropic drugs: Animal models for studying effects on cognition. Drug Develop Res 4: p 567-576, 1984], and in particular, an oxidative metabolism disorder in brain by NaNO₂ is intimately connected with memory disturbance owing to cholinergic blockade. Therefore, if a delay is observed in the time for NaNO₂-mediated death induction upon drug treatment, it can be considered as one of indications reflecting memory-improving effect of the drug.

1) Experimental Method

An extract of Liriopsis tuber (fraction T) was administered (10 mg/kg) via P.O. to male mice (20 g) and after 60 min, NaNO₂ (250 mg/kg) was administered via s.c. Time period until breath stops was measured, and breath-duration time was compared with that of control group to evaluate a memory-improving effect.

2) Experimental Result

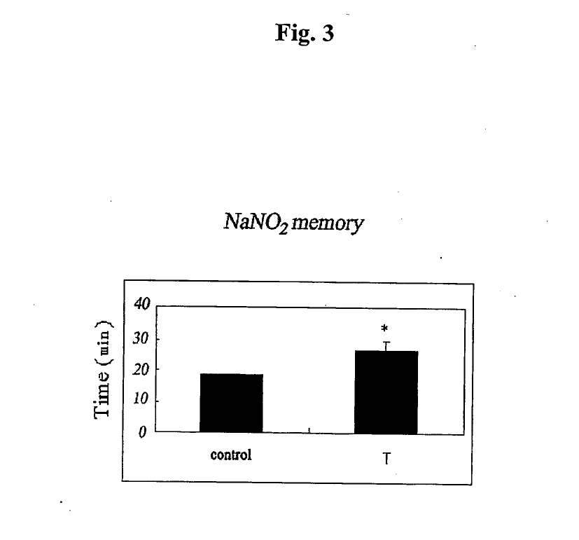

As can be seen from FIG. 3, pretreatment with the Liriopsis tuber extract (fraction T) (10 mg/kg, P.O.) induced 45% increase in the time for death induction due to the brain metabolism disorder by NaNO₂, revealing a memory improving effect of the Liriopsis tuber extract.

### EXPERIMENTAL EXAMPLE 3

### Passive Avoidance Test

1) Experimental Method

Male mice (20 g) were administered with Liriopsis tuber extracts (fractions T, A, C, CM and M) via P.O. route for three days (10 mg/kg per day), and a passive avoidance test was performed using Gemini Avoidance System (San Diego Instruments, USA). The experiment was carried out basically according to Kumar et al. method with some modifications [Kumar, V., Singh, P. N., Muruganandan, A. V., Bhattacharya. Effect of Indian Hypericum perforatum Linn on animal models of cognitive dysfunction, J. Ethnophannacology 72, p 119-128, 2000].

In the case of training experiment on the first day, the mice were put into a light box, subjected to acclimation for 300 sec and then allowed to move into a dark box by making the door to be opened automatically. Upon moving into the dark box, electric stimulus of 0.3 mA was given for 1 sec. Scopolamine was administered (1 mg/kg, i.p.) immediately after termination of the training session. In the case of test experiment conducted after 24 hr, the mice were subjected to acclimation for 300 sec in the light box, the door was opened and the mice were allowed to move into the dark box. Time for moving into the dark box was measured. On the second day, electric stimulus was not given. If mouse stayed without moving into the dark box for 500 sec, maximum score, 500 sec was given.

2) Experimental Result

In the experiment on the first day, there was no significant difference among the groups. In the test experiment on the second day, it has been found that mice with scopolamine-induced dementia have decreased memory by 83% to the control group. However, the mice administered with Liriopsis tuber extracts (fractions T, A, C and M) for 3 days, restored memory up to 33%, 32%, 45% and 158%, respectively, against memory disorder due to scopolamine

### EXPERIMENTAL EXAMPLE 4

### Ex Vivo Cholinesterase Assay

1) Experimental Method

Male SD rats were orally administered with an extract of Liriopsis tuber (10 mg/kg) and after 60 min, brain was taken out and then hippocampus was separated, resuspended with an isolation buffer containing 50 mM Tris HCl, 1 mM EDTA, 1 mM EGTA, 150 mM NaCl, 1% Triton X-100, 0.5 mM PMSF, 1 mM Na₃VO₄, 1 μg/ml leupeptin and 1 μg/ml aprotinin, and homogenized with a Potter-Elvehjem homogenizer. The insoluble material was removed by centrifugation for 20 min (10,000×g) at 4° C. Activity of cholinesterase was determined by Ellman et al. method [Ellman, G. L., Courtney, K. D., Andres, V., Featherstone, R. M. A new and rapid colorimetric determination of acetylcholinesterase activity, Biochem. Pharmacol. 7, 88-95, 1961]. Buffer I (100 mM phosphate, pH 8.0) 3 ml, 75 mM acetylthiocholine iodide 0.2 ml and buffered Elimans reagent (DTNB 10 mM, NaHCO₃ 15 mM) 0.1 ml were mixed and reacted at 25° C. for 10 min. To this reaction mixture, hippocampus lysate 20 μl was added and absorbance was determined by 30 sec interval. Percentage inhibition was calculated via comparing with the control group.

2) Experimental Result

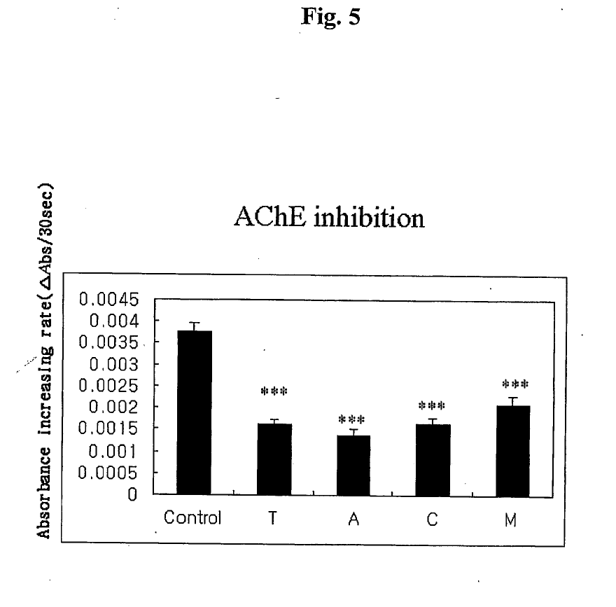

The administration of extracts of Liriopsis tuber (fractions T, A, C and M) inhibited cholinesterase activity by 56%, 64%, 56% and 44%, respectively (FIG. 5).

### EXPERIMENTAL EXAMPLE 5

### Effect on ERK I/II Activity

1) Experimental Method

Effect of extracts of Liriopsis tuber on the activity of ERK (Extracellular signal-regulated kinase) I/II was determined as follows.

Each fractions of Liriopsis tuber extracts was orally administered (10 mg/kg), hippocampus was separated after 1 hr and put into the isolation buffer as prepared in the Experimental Example 4 and homogenized with a Potter-Elvehjem homogenizer, and ERK I/II activity was determined based on SDS-PAGE (sodium dodecyl sulfate polyacrylamide gel electrophoresis) and Western blotting as described below.

For the PAGE, 30 μl samples containing an equal amount of protein were applied. Five folds Laemmli's sample buffer was added, boiled in water for 5 min and sample thus obtained was loaded and subjected to electrophoresis at 100V. SDS-PAGE condition: 7.5% resolving gel. After carrying out SDS-PAGE, proteins were allowed to transfer to nitrocellulose membrane for 1 hr at 100V using Mini Trans-Blot Electrophoretic Transfer Cell (Bio-Rad). The nitrocellulose membrane was soaked in blocking solution prepared by dissolving 5% skim milk in PBS solution containing 0.1% Tween 20 (PBS-T), and incubated overnight in 4° C. refrigerator. Solution of primary antibody (anti ERK I/II Ab and anti-phospho ERIC I/II Ab; New England Biolab, USA) diluted with PBS-T to 1:1000 was added to the nitrocellulose membrane and reacted for 1 hr. The nitrocellulose membrane was washed with PBS-T once for 15 min, three times for 5 min, and solution of secondary antibody (horseradish peroxidase-linked goat anti-rabbit IgG (Pierce)) diluted with PBS-T to 1:1000 was added and reacted for 40 min. ERK II (42 kDa) protein band was identified by enhanced chemiluminescence (ECL, Pierce) (Harlow E. and Lane D., Antibodies: A laboratory manual, 726, 1988).

2) Experimental Result

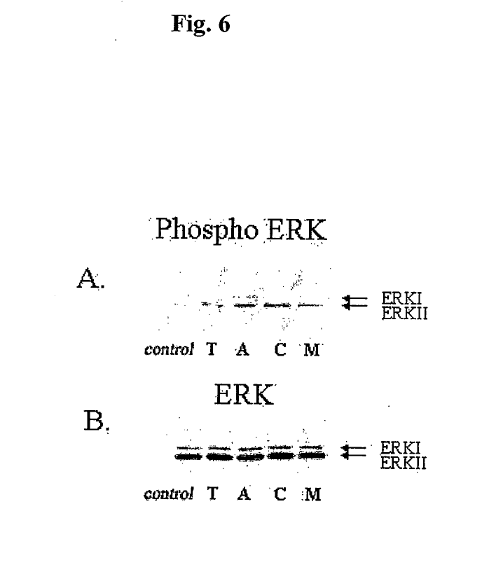

As a result, the amount of ERK I/II activated via phosphorylation (phospho-ERK I/II) was seen to be remarkably increased upon administration of Liriopsis tuber extracts (fractions T, A, C and M) when compared to the control group (FIG. 6A). On the other hand, protein content of ERK I/II was nearly equal in both control group and administered groups (FIG. 6B). Based on this result, it can be confirmed that the extracts of Liriopsis tuber (fractions T, A, C and M) induce memory-improving action via activating ERIC I/II of hippocampus in rat.

### EXPERIMENTAL EXAMPLE 6

### Effect on the Activity of Insulin Receptor

Activation of insulin receptor plays an important role on memory formation. As the activation of insulin receptor is induced via phosphorylation of tyrosine residue in β subunit thereof, the effect of a Liriopsis tuber extract on the activation of insulin receptor was analyzed as follows.

1) Experimental Method

Each fraction of the extracts of Liriopsis tuber was orally administered (10 mg/kg concentration) to rats and after 1 hr, hippocampus was separated, placed in 4 folds of the isolation buffer as prepared in Experimental Example 4 to the weight of hippocampus and homogenized using a Potter-Elvehjem homogenizer. Then, activity of insulin receptor was determined as described below by immunoprecipitation reaction and SDS-PAGE electrophoresis, and Western blotting.

Homogenized hippocampus lysate 1000 was mixed with buffer (0.5M NaCl, 1% NP-40, 10% deoxycholate, 0.1% SDS) 100 μl, reacted at 4° C. for 1 hr, and the solubilized proteins were isolated by centrifugation. To the supernatant, insulin receptor antibody (Transduction laboratories) (5 μl) was added. The reaction mixture was allowed to rotate using a rotor for 1 hr and protein A Sepharose (20 μl) was added and reacted at 4 ° C. for 1 hr using the rotor. The immune complex was precipitated by centrifugation. The pellets were washed with washing buffers A (0.01M Tris, pH 7.4, 1M NaCl, 1% Nonidet P-40), B (0.01M Tris, pH 7.4, 0.1M NaCl, 0.01M EDTA, 1% Nonidet P-40, 0.3% SDS) and C (0.01M Tris, pH 7.4, and 1% Nonidet P-40), sequentially. The final pellets were solubilized with. Laemmli's sample buffer containing 100 mM dithiothreitol, boiled for 5 min, centrifuged in a microcentrifuge, and the supernatant was subjected to SDS-PAGE: 7.5% resolving gel. After electrophoresis,'Western blotting was conducted as described in Experimental Example 3, and phosphorylation of tyrosine residue on insulin receptor β subunit was observed using phosphotyrosine Ab (Transduction laboratories) as an antibody.

2) Experimental Result

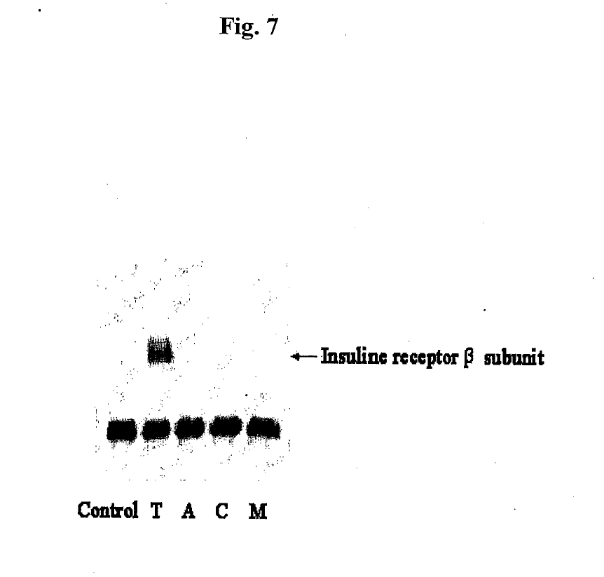

As it can be seen from FIG. 7, the fraction T of the Liriopsis tuber extract noticeably activated insulin receptor and fraction C as well exhibited remarkable effect on activating insulin receptor compared to the control group. Accordingly, it can be concluded that activation of insulin receptor plays a critical role in the memory-improving effect by fractions T and C confirmed in said Experimental example 3.

### FORMULATION EXAMPLE 1

### Tablets

Tablets of the following components were formulated according to conventional manufacturing method for tablets.

1-1. Tablet composition

| | | |
|---|---|---|
| Methanol extract of Liriopsis tuber | 500.0 | mg |
| Lactose | 500.0 | mg |
| Talc | 5.0 | mg |
| Magnesium stearate | 1.0 | mg |

1-2. Tablet composition

| | | |
|---|---|---|
| Chloroform fraction of methanol extract of Liriopsis tuber | 50.0 | mg |
| Lactose | 50.0 | mg |
| Talc | 0.5 | mg |
| Magnesium stearate | 0.1 | mg |

1-3. Tablet composition

| | | |
|---|---|---|
| Methanol fraction of methanol extract of Liriopsis tuber | 50.0 | mg |
| Lactose | 50.0 | mg |
| Talc | 0.5 | mg |
| Magnesium stearate | 0.1 | mg |

1-4. Tablet composition

| | | |
|---|---|---|
| Methanol-insoluble fraction of methanol extract | 50.0 | mg |
| of Liriopsis tuber | | |
| Lactose | 50.0 | mg |
| Talc | 0.5 | mg |
| Magnesium stearate | 0.1 | mg |

### FORMULATION EXAMPLE 2

### Capsules

Based on the following composition, capsules were prepared by the following process. The extract of Liriopsis tuber was screened, mixed with excipient, filled into gelatin capsule to prepare capsules.

2-1. Capsule composition

| | | |
|---|---|---|
| Methanol extract of Liriopsis tuber | 500.0 | mg |
| Starch 1500 | 10.0 | mg |
| Magnesium stearate BP | 100.0 | mg |

2-2. Capsule composition

| | | |
|---|---|---|
| Chloroform fraction of methanol extract of Liriopsis tuber | 50.0 | mg |
| Starch 1500 | 1.0 | mg |
| Magnesium stearate BP | 10.0 | mg |

2-3. Capsule composition

| | | |
|---|---|---|
| Methanol fraction of methanol extract of Liriopsis tuber | 50.0 | mg |
| Starch 1500 | 1.0 | mg |
| Magnesium stearate BP | 10.0 | mg |

2-4. Capsule composition

| | | |
|---|---|---|
| Methanol insoluble fraction of methanol extract | 50.0 | mg |
| of Liriopsis tuber | | |
| Starch 1500 | 1.0 | mg |
| Magnesium stearate BP | 10.0 | mg |

### FORMULATION EXAMPLE 3

### Syrups

Based on the following composition, syrups were prepared as follows. Sugar was dissolved in purified water and then paraoxybenzoate, paraoxypropylbenzoate and Liriopsis tuber extract were added, dissolved at 60° C., cooled and purified water was added to 150 ml.

3-1. Syrup composition

| | | |
|---|---|---|
| Methanol extract of Liriopsis tuber | 5.0 | g |
| Sugar | 95.1 | g |
| Paraoxybenzoate | 80.0 | mg |
| Paraoxypropylbenzoate | 16.0 | mg |
| Purified water | to 150 | ml |

3-2. Syrup composition

| | | |
|---|---|---|
| Chloroform fraction of methanol extract of Liriopsis tuber | 50.0 | mg |
| Sugar | 95.1 | g |
| Paraoxybenzoate | 80.0 | mg |
| Paraoxypropylbenzoate | 16.0 | mg |
| Purified water | to 150 | ml |

3-3. Syrup composition

| | | |
|---|---|---|
| Methanol fraction of methanol extract of Liriopsis tuber | 50.0 | mg |
| Sugar | 95.1 | g |
| Paraoxybenzoate | 80.0 | mg |
| Paraoxypropylbenzoate | 16.0 | mg |
| Purified water | to 150 | ml |

3-4. Syrup composition

| | | |
|---|---|---|
| Methanol insoluble fraction of methanol extract of Liriopsis | 50.0 | mg |
| tuber | | |
| Sugar | 95.1 | g |
| Paraoxybenzoate | 80.0 | mg |
| Paraoxypropylbenzoate | 16.0 | mg |
| Purified water | to 150 | ml |

### FORMULATION EXAMPLE 4

### Solutions

The following components were formulated by conventional process, and filled into brown bottles to prepare solutions.

4-1. Solution composition

| | | |
|---|---|---|
| Methanol extract of Liriopsis tuber | 500.0 | mg |
| Isomerized sugar | 20.0 | g |
| Antioxidant | 5.0 | mg |
| Methyl paraoxybenzoate | 2.0 | mg |
| Purified water | to 100.0 | ml |

4-2. Solution composition

| | | |
|---|---|---|
| Chloroform fraction of methanol extract of Liriopsis tuber | 500.0 | mg |
| Isomerized sugar | 20.0 | g |
| Antioxidant | 5.0 | mg |
| Methyl paraoxybenzoate | 2.0 | mg |
| Purified water | to 100.0 | ml |

4-3. Solution composition

| | | |
|---|---|---|
| Methanol fraction of methanol extract of Liriopsis tuber | 500.0 | mg |
| Isomerized sugar | 20.0 | g |
| Antioxidant | 5.0 | mg |
| Methyl paraoxybenzoate | 2.0 | mg |
| Purified water | to 100.0 | ml |

4-4. Solution composition

| | | |
|---|---|---|
| Methanol insoluble fraction of methanol extract of Liriopsis | 500.0 | mg |
| tuber | | |
| Isomerized sugar | 20.0 | g |
| Antioxidant | 5.0 | mg |
| Methyl paraoxybenzoate | 2.0 | mg |
| Purified water | to 100.0 | ml |

### FORMULATION EXAMPLE 5

### Powders

The following components were mixed, filled in bag and sealed to prepare powders by conventional method for powders.

5-1. Powder composition

| | |
|---|---|
| Methanol extract of Liriopsis tuber | 50.0 mg |
| Lactose | 100.0 mg |
| Talc | 5.0 mg |

5-2. Powder composition

| | |
|---|---|
| Chloroform fraction of methanol extract of Liriopsis tuber | 50.0 mg |
| Lactose | 100.0 mg |
| Talc | 5.0 mg |

5-3. Powder composition

| | |
|---|---|
| Methanol fraction of methanol extract of Liriopsis tuber | 50.0 mg |
| Lactose | 100.0 mg |
| Talc | 5.0 mg |

5-4. Powder composition

| | |
|---|---|
| Methanol insoluble fraction of methanol extract of Liriopsis | 50.0 mg |
| tuber | |
| Lactose | 100.0 mg |
| Talc | 5.0 mg |

### FORMULATION EXAMPLE 6

### Injections

The following components were filled in 2.0 ml amples, subjected to sterilization to prepare injections by conventional process for injections.

6-1. Injection composition

| | | |
|---|---|---|
| Methanol extract of Liriopsis tuber | 50.0 | mg |
| Antioxidant | 1.0 | mg |
| Tween 80 | 1.0 | mg |
| Distilled water for injection | to 2.0 | ml |

6-2. Injection composition

| | | |
|---|---|---|
| Chloroform fraction of methanol extract of Liriopsis tuber | 50.0 | mg |
| Antioxidant | 1.0 | mg |
| Tween 80 | 1.0 | mg |
| Distilled water for injection | to 2.0 | ml |

6-3. Injection composition

| | | |
|---|---|---|
| Methanol fraction of methanol extract of Liriopsis tuber | 50.0 | mg |
| Antioxidant | 1.0 | mg |
| Tween 80 | 1.0 | mg |
| Distilled water for injection | to 2.0 | ml |

6-4. Injection composition

| | | |
|---|---|---|
| Methanol insoluble fraction of methanol extract of Liriopsis | 50.0 | mg |
| tuber | | |
| Antioxidant | 1.0 | mg |
| Tween 80 | 1.0 | mg |
| Distilled water for injection | to 2.0 | ml |

### FORMULATION EXAMPLE 7

### Preparation of Sunsik

Brown rice, barley, glutinous rice and Job's tear were gelatinized, dried, parched and ground to 60 mesh powder according to conventional method. Black bean, black sesame and Perilla japonica as well were boiled, dried, parched and ground to 60 mesh powder by conventional method. Grains, seed-fruits and dried extract of Liriopsis tuber prepared as described above were combined by the following ratio to prepare granules.

### 7-1. Preparation Example of Sunsik

Grains: brown rice 30 w/w %, Job's tear 15%, barley 20 w/w % and glutinous rice 9% Seed-fruits: Perilla japonica 7 w/w %, black bean 8 w/w % and black sesame 7 w/w % Dried powder of methanol extract of Liriopsis tuber: 3 w/w %, Ganoderma Lucidum (FR) karst 0.5 w/w % and Rehmannia glutinosa 0.5 w/w %

### 7-2. Preparation Example of Sunsik

Grains: brown rice 30 w/w %, Job's tear 15%, barley 20 w/w % and glutinous rice 9% Seed-fruits: Perilla japonica 7 w/w %, black bean 8 w/w % and black sesame 7 w/w % Dried powder of chloroform fraction in methanol extract of Liriopsis tuber: 3 w/w %, Ganoderma Lucidum (FR) karst 0.5 w/w % and Rehmannia glutinosa 0.5 w/w %

### 7-3. Preparation Example of Sunsik

Grains: brown rice 30 w/w %, Job's tear 15%, barley 20 w/w % and glutinous rice 9% Seed-fruits: Perilla japonica 7 w/w %, black bean 8 w/w % and black sesame 7 w/w % Dried powder of methanol fraction in methanol extract of Liriopsis tuber: 3 w/w %, Ganoderma Lucidum (FR) karst 0.5 w/w % and Rehmannia glutinosa 0.5 w/w %

### 7-4.Preparation Example of Sunsik

Grains: brown rice 30 w/w %, Job's tear 15%, barley 20 w/w % and glutinous rice 9% Seed-fruits: Perilla japonica 7 w/w %, black bean 8 w/w % and black sesame 7 w/w % Dried powder of methanol-insoluble fraction from methanol extract of Liriopsis tuber: 3 w/w %, Ganoderma Lucidum (FR) karst 0.5 w/w % and Rehmannia glutinosa 0.5 w/w %

### INDUSTRIAL APPLICABILITY

The composition comprising an extract of Liriopsis tuber of the present invention exhibits effects of preventing and treating neurodegenerative diseases caused by brain cell damage and of improving memory and accordingly, it can be used for protecting brain cells in persons under the risk of brain damage by various environmental stress and for improving memory in persons suffering from memory decline including dementia.

1-47. (canceled)

48. A method for protecting brain cells and improving the memory of a patient, said method comprising inhibiting nerve cell depolarization by AMPA by administering a therapeutically effective amount of a medicament to a patient in need thereof, said medicament comprising an extract of Liriopsis tuber in an amount from about 0.1 mg/kg to about 500 mg/kg.

49. The method of claim 48, wherein said extract of Liriopsis tuber is derived by a first extraction process comprising extracting the Liriopsis tuber with a solvent selected from the group consisting of C1-4 lower alcohols or a mixture of said lower alcohols with water, acetone, chloroform, methylene chloride, ether, and ethyl acetate to obtain a rough extract.

50. The method of claim 49, wherein the first extraction process is followed by a second extraction process comprising dissolving the rough extract in a mixture of C1-4 lower alcohol and water, adjusting the pH with an acid to a range of 2-4, and extracting with chloroform to obtain a chloroform soluble fraction and a chloroform insoluble aqueous fraction.

51. The method of claim 50, wherein the second extraction process is followed by a third extraction process comprising adjusting the chloroform insoluble aqueous fraction with ammonium hydroxide to adjust a pH range of 9-12 and extracting with a chloroform-methanol mixture to obtain a chloroform-methanol soluble fraction and a methanol-chloroform insoluble aqueous fraction.

52. The method of claim 51, wherein the third extraction process is followed by a fourth extraction process comprising extracting the methanol-chloroform insoluble fraction with methanol to obtain a methanol soluble fraction and a water soluble fraction.

53. The method of claim 48, wherein said Liriopsis tuber is selected from the group consisting of Liriope platyphylla, Ophiopogon japonicus, Ophiopogon stolonifer, Mondo japonicum, and Liriope spicata.

54. A method for protecting brain cells and improving the memory of a patient, said method comprising inhibiting cholinesterase activity in the brain of a patient by administering a therapeutically effective amount of a medicament to a patient in need thereof, said medicament comprising an extract of Liriopsis tuber in an amount from about 0.1 mg/kg to about 500 mg/kg.

55. The method of claim 54, wherein said extract of Liriopsis tuber is derived by a first extraction process comprising extracting the Liriopsis tuber with a solvent selected from the group consisting of C1-4 lower alcohols or a mixture of said lower alcohols with water, acetone, chloroform, methylene chloride, ether, and ethyl acetate to obtain a rough extract.

56. The method of claim 55, wherein the first extraction process is followed by a second extraction process comprising dissolving the rough extract in a mixture of C1-4 lower alcohol and water, adjusting the pH with an acid to a range of 2-4, and extracting with chloroform to obtain a chloroform soluble fraction and a chloroform insoluble aqueous fraction.

57. The method of claim 56, wherein the second extraction process is followed by a third extraction process comprising adjusting the chloroform insoluble aqueous fraction with ammonium hydroxide to adjust a pH range of 9-12 and extracting with a chloroform-methanol mixture to obtain a chloroform-methanol soluble fraction and a methanol-chloroform insoluble aqueous fraction.

58. The method of claim 57, wherein the third extraction process is followed by a fourth extraction process comprising extracting the methanol-chloroform insoluble fraction with methanol to obtain a methanol soluble fraction and a water soluble fraction.

59. The method of claim 54, wherein said Liriopsis tuber is selected from the group consisting of Liriope platyphylla, Ophiopogon japonicus, Ophiopogon stolonifer, Mondo japonicum, and Liriope spicata.

60. A method for protecting brain cells and improving the memory of a patient, said method comprising phosphorylating a protein in the brain of a patient by administering a therapeutically effective amount of a medicament to a patient in need thereof, said medicament comprising an extract of Liriopsis tuber in an amount from about 0.1 mg/kg to about 500 mg/kg.

61. The method of claim 60 wherein the protein is an insulin receptor.

62. The method of claim 60 wherein the protein is ERK I/II.

63. The method of claim 60, wherein said extract of Liriopsis tuber is derived by a first extraction process comprising extracting the Liriopsis tuber with a solvent selected from the group consisting of C1-4 lower alcohols or a mixture of said lower alcohols with water, acetone, chloroform, methylene chloride, ether, and ethyl acetate to obtain a rough extract.

64. The method of claim 63, wherein the first extraction process is followed by a second extraction process comprising dissolving the rough extract in a mixture of C1-4 lower alcohol and water, adjusting the pH with an acid to a range of 2-4, and extracting with chloroform to obtain a chloroform soluble fraction and a chloroform insoluble aqueous fraction.

65. The method of claim 64, wherein the second extraction process is followed by a third extraction process comprising adjusting the chloroform insoluble aqueous fraction with ammonium hydroxide to adjust a pH range of 9-12 and extracting with a chloroform-methanol mixture to obtain a chloroform-methanol soluble fraction and a methanol-chloroform insoluble aqueous fraction.

66. The method of claim 65, wherein the third extraction process is followed by a fourth extraction process comprising extracting the methanol-chloroform insoluble fraction with methanol to obtain a methanol soluble fraction and a water soluble fraction.

67. The method of claim 60, wherein said Liriopsis tuber is selected from the group consisting of Liriope platyphylla, Ophiopogon japonicus, Ophiopogon stolonifer, Mondo japonicum, and Liriope spicata.
